(19) United States
(12) Patent Application Publication
(10) Pub. No.: US 2001/0021533 A1
(43) Pub. Date: Sep. 13, 2001

(54) Multi-partite ligands and methods of identifying and using same

(75) Inventor: Daniel S. Sem, San Diego, CA (US)

Correspondence Address: CAMPBELL & FLORES LLP, 7th Floor, 4370 La Jolla Village Drive, San Diego, CA, 92122, US

(73) Assignee: Triad Biotechnology, Inc.

(21) Appl. No.: 09/765,693

(22) Filed: Jan. 19, 2001

Related U.S. Application Data
(62) Division of application No. 09/083,537, filed on May 21, 1998.

(51) Int. Cl. G01N 33/543

(52) U.S. Cl. 436/518

(57) ABSTRACT

The invention provides methods for generating a library of bi-ligands, comprising (a) determining a common ligand to a conserved site in a receptor family; (b) attaching an expansion linker to the common ligand, wherein the expansion linker has sufficient length and orientation to direct a second ligand to a specificity site of a receptor in the receptor family, to form a module; and (c) generating a population of bi-ligands comprising a plurality of identical modules attached to variable second ligands. The invention also provides methods for identifying a bi-target ligand to a receptor by combining a first bi-ligand to a first receptor in a receptor family and a second bi-ligand to a second receptor in the receptor family. The invention additionally provides bi-ligands and bi-target ligands.

### BACKGROUND OF THE INVENTION

### FIELD OF THE INVENTION

The present invention relates generally to medicinal chemistry and more specifically to agents which bind to more than one site on an enzyme.

One of the major scientific undertakings of recent years has been the identification of genetic information with the ultimate goal being the determination of the entire genome of an organism and its encoded genes, termed genomic studies. One of the most ambitious of these genomic projects has been the Human Genome Project, with the goal of sequencing the entire human genome. Recent advances in sequencing technology have led to a rapid accumulation of genetic information, which is available in both public and private databases. These newly discovered genes as well as those genes soon to be discovered provide a rich resource of potential targets for the development of new drugs.

Two general approaches have traditionally been used for drug discovery, screening for lead compounds and structure-based drug design. Both approaches have advantages and disadvantages, with the most significant disadvantage being the laborious and time-consuming nature of using these approaches to discovery of new drugs.

Drug discovery and development based on screening for lead compounds involves generating a pool of candidate compounds, often using combinatorial chemistry in which compounds are synthesized by combining chemical groups to generate a large number of diverse candidate compounds. The candidate compounds are screened with a drug target of interest to identify lead compounds. However, the screening process to identify a lead compound that binds to the target can be laborious and time consuming. Moreover, the lead compound often has to be further modified and screened to identify a compound that functions as a potential drug having desired activity toward a target of interest.

Structure-based drug design is an alternative approach to identifying candidate drugs. Structure-based drug design uses three-dimensional structural data, both calculated and predicted, of the drug target as a template to model compounds that inhibit or otherwise interfere with critical residues that are required for activity of the drug target. The compounds identified as potential drug candidates using structural modeling are used as lead compounds for the development of candidate drugs that exhibit a desired activity toward the drug target.

Identifying compounds using structure-based drug design can be advantageous over the screening approach in that modifications to the compound can often be predicted based on the modeling studies. However, obtaining structures of relevant drug targets and their complexes with compounds is extremely time consuming and laborious, often taking years to accomplish. The long time period required to obtain structural information useful for developing drug candidates is particularly limiting with regard to the growing number of newly discovered genes, which are potential drug targets, identified in genomics studies.

Despite the time-consuming and laborious nature of these approaches to drug discovery, both screening for lead compounds and structure-based drug design have led to the identification of a number of useful drugs. However, even with drugs useful for treating particular diseases, many of the drugs have unwanted toxicity or side effects. For example, in addition to binding to the drug target in a pathogenic organism or cancer cell, in some cases the drug also binds to an analogous protein in the patient being treated with the drug, which can result in toxic or unwanted side effects. Therefore, drugs that have high affinity and specificity for a target are particularly useful because administration of a more specific drug at lower dosages will minimize toxicity and side effects.

In addition to drug toxicity and side effects, a number of drugs that were previously highly effective at treating certain diseases have become less effective during prolonged clinical use due to the development of resistance. Drug resistance has become increasingly problematic, particularly with regard to administration of antibiotics. A number of pathogenic organisms have become resistant to several drugs due to prolonged clinical use and, in some cases, have become almost totally resistant to currently available drugs (MMWR Morb. Mortal. Wkly. Rep. 46:624-626 (1997)). Furthermore, certain types of cancer develop resistance to cancer therapeutic agents. Therefore, drugs that are inherently refractile to the development of resistance would be particularly desirable for treatment of a variety of diseases.

Thus, there exists a need to rapidly and efficiently identify ligands that bind to a drug target that will remain effective during prolonged clinical use. In addition, there exists a need to rapidly identify drugs targeting genes that are newly identified from genomic studies. The present invention satisfies these needs and provides related advantages as well.

### SUMMARY OF THE INVENTION

The invention provides methods for generating a library of bi-ligands, comprising (a) determining a common ligand to a conserved site in a receptor family; (b) attaching an expansion linker to the common ligand, wherein the expansion linker has sufficient length and orientation to direct a second ligand to a specificity site of a receptor in the receptor family, to form a module; and (c) generating a population of bi-ligands comprising a plurality of identical modules attached to variable second ligands. The invention also provides methods for identifying a bi-target ligand to a receptor, comprising (a) identifying a first bi-ligand to a first receptor in a receptor family, wherein the bi-ligand comprises a common ligand to a conserved site in a receptor family and a first specificity ligand to the first receptor; (b) identifying a second bi-ligand to a second receptor in the receptor family, wherein the bi-ligand comprises the common ligand and a second specificity ligand to the second receptor; and (c) generating a bi-target ligand comprising the common ligand, the first specificity ligand and the second specificity ligand, whereby the bi-target ligand can bind to the first receptor and the second receptor. The invention also provides bi-ligands and bi-target ligands.

### BRIEF DESCRIPTION OF THE DRAWINGS

FIG. 1 shows a diagram representing bi-ligands bound to specific receptors. The bi-ligand contains three components, a common ligand, a specificity ligand and an expansion linker. The common ligand, which binds to a conserved site in a receptor family, is designated by a pentagon. The specificity ligand binds to a specificity site on the receptor and is depicted as a triangle, square, circle and star for drugs 1 through 4, respectively. The expansion linker, indicated by two lines, bridges the common ligand and specificity ligand in an orientation allowing both the common ligand and specificity ligand to bind simultaneously to the respective conserved site and specificity site on the receptor.

FIG. 2 shows a diagram of a bi-target ligand and four bi-ligands bound to specific receptors. The bi-target ligand contains four components, a common ligand, two specificity ligands, and an expansion linker. The common ligand, which binds to a conserved site in a receptor family, is designated by a pentagon. The specificity ligands of the bi-target ligand, designated by a square and a triangle, bind to targets 1 and 2, respectively. The expansion linker, indicated by three lines, bridges the common ligand and the specificity ligands in an orientation allowing the common ligand and one of the specificity ligands to bind simultaneously to its specific target. The bi-target ligand depicted can bind to the common site and specificity site on target 1 or the common site and specificity site on target 2.

FIG. 3 shows a representation of (A) “perfect C₂ symmetry” and (B) “approximate C₂ symmetry” relationship between points of attachment for specificity ligands to a morpholine substitute expansion linker. Perfect C₂ symmetry means that, if the expansion linker is rotated 180° about the axis defined by the common ligand attachment to the expansion linker, then the positions of the two specificity ligands are exactly swapped. This swapping is such that if the two specificity ligands were identical, the overall conformation of the molecule would be indistinguishable from the conformation before the rotation. Approximate C₂ symmetry means that, if the expansion linker is rotated 180° about the axis defined by the common ligand attachment to the expansion linker, then the positions of the two specificity ligands are approximately swapped. This swapping is such that a given specificity ligand occupies substantially the same position in space as the other specificity ligand did prior to the rotation, within about 5 Å.

### DETAILED DESCRIPTION OF THE INVENTION

The invention provides multi-partite ligands and methods for identifying a multi-partite ligand that binds to at least two sites on a receptor. The methods are applicable for the identification of ligands to a desired target receptor. Such ligands can be developed as potential drug candidates. The methods are advantageous in that they use a common ligand that binds to a conserved site in a receptor family as a starting compound to identify a ligand that binds with high specificity and affinity to a receptor target. The methods involve determining molecular modules in an object-oriented manner to build multi-partite ligands that are multi-functional. The molecular modules, or objects, have defined attributes and functions, such as binding to a receptor, and the molecular modules are combined in different ways to generate multi-partite ligands with defined attributes and functions. Object-oriented design processes have been previously applied to software development (Fowler and Scott, in UML Distilled: Applying the Standard Object Modeling Language, Addison-Wesley, Berkeley, (1997); and Booch, in Object Solutions: Managing the Object Oriented Project Addison-Wesley, Menlo Park, (1996)).

A bi-ligand that binds to two sites on a target receptor is generated by attaching a second ligand, which binds to a specificity site on the receptor, to a common ligand that binds to a conserved site in a receptor family. The common ligand and specificity ligand are bridged by a linker, which is attached to the common ligand such that the specificity ligand is positioned and orientated for optimized binding to a site specific for the receptor. This orientation and positioning is determined by obtaining limited structural information on the target receptor complexed to the common ligand that is sufficient to identify a site on the common ligand that is oriented towards a specificity site on the receptor. Such a multi-partite ligand with an expansion linker, which orients and positions the specificity ligand relative to the common ligand for optimized binding, is more likely to exhibit high affinity for a receptor and to have specificity for a particular target receptor.

Furthermore, a second specificity ligand having specificity for a second receptor in the receptor family can be identified and combined with the first bi-ligand. Such a tri-ligand incorporates the common ligand that binds to multiple members of a receptor family and two specificity ligands that specifically bind to two receptor targets. Thus, a single, bi-target ligand is generated that is capable of binding to two different target receptors. Such a bi-target ligand is inherently refractile to the development of resistance to the ligand during clinical use. Since resistance to a drug can develop following mutations in the target receptor, a single bi-target ligand capable of binding to two receptors requires mutations in two different genes, thus decreasing the probability of developing resistance to the ligand.

As used herein, the term “ligand” refers to a molecule that can selectively bind to a receptor. The term selectively means that the binding interaction is detectable over non-specific interactions by a quantifiable assay. A ligand can be essentially any type of molecule such as an amino acid, peptide, polypeptide, nucleic acid, carbohydrate, lipid, or any organic compound. Moreover, derivatives, analogues and mimetic compounds are also intended to be included within the definition of this term, including the addition of metals or other inorganic molecules. A ligand can be multi-partite, comprising multiple ligands capable of binding to different sites on one or more receptors. The ligand components of a multi-partite ligand are joined together by an expansion linker. The term ligand therefore refers both to a molecule capable of binding to a receptor and to a portion of such a molecule, if that portion of a molecule is capable of binding to a receptor.

As used herein, the term “common ligand” refers to a ligand that binds to a conserved site in a receptor family. As used herein, “natural common ligand” refers to a ligand that is found in nature and binds to a common site in a receptor family. As used herein, the term “specificity ligand” refers to a ligand that, when attached to a common ligand, binds to a specificity site on a receptor that is proximal to the conserved site.

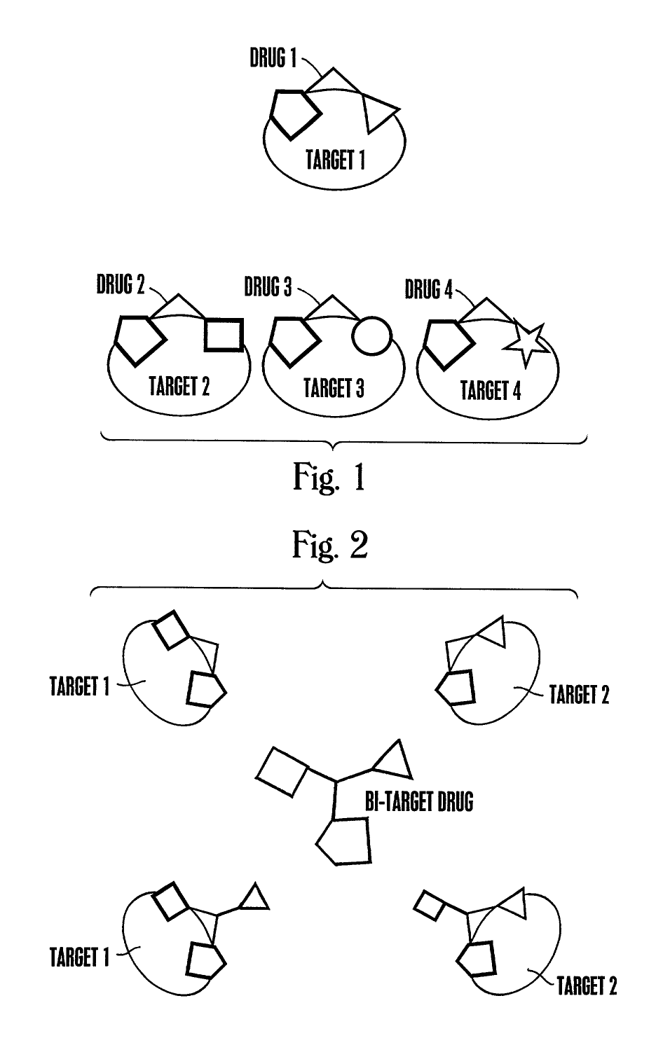

As used herein, the term “bi-ligand” refers to a ligand comprising two ligands, both of which can bind to a receptor when tethered by an expansion linker. One of the ligands of a bi-ligand is a common ligand that binds to a conserved site in a receptor family. The second ligand of a bi-ligand is a specificity ligand capable of binding to a site that is specific for a given member of a receptor family when joined to a common ligand. The common ligand and specificity ligand are joined together by an expansion linker. A depiction of bi-ligands is shown in FIG. 1.

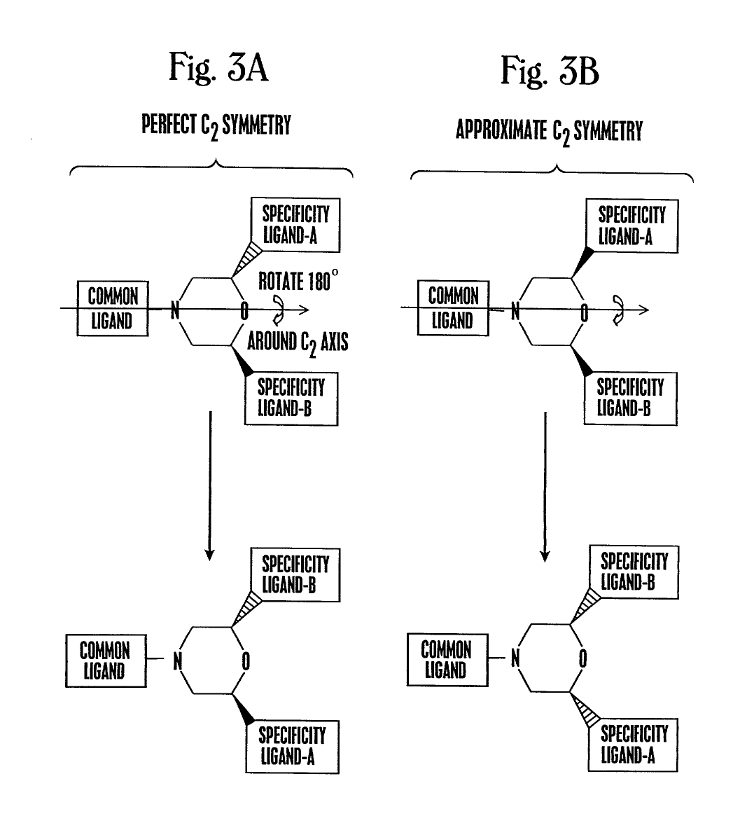

As used herein, the term “bi-target ligand” refers to a ligand comprising three distinct ligands. One of the ligands of a bi-target ligand is a common ligand that binds to a conserved site in a receptor family. The other two ligands are specificity ligands. One of the specificity ligands of a bi-target ligand binds to a specificity site of a receptor in a receptor family. The second specificity ligand of a bi-target ligand binds to a specificity site of a different member of the same receptor family. A bi-target ligand is therefore capable of binding to two different target receptors in the same family. The common ligand and specificity ligands are joined together by an expansion linker. A depiction of a bi-target ligand is shown in FIG. 2.

As used herein, the term “expansion linker” refers to a chemical group that is capable of linking two ligands that bind to the same receptor. An expansion linker is used to bridge a common ligand to one or more specificity ligands. An expansion linker can be optimized to provide positioning and orientation of the specificity ligand relative to the common ligand such that the common ligand and specificity ligand are positioned to bind to their respective conserved site and specificity site on a receptor. It is advantageous to have the expansion linker comprise a molecule providing C2 symmetry or approximate C2 symmetry in the case where the expansion linker is ultimately to be used in constructing a bi-target ligand. However, this is not a required property for bi-ligand molecules.

As used herein, “perfect C2 symmetry”, when used in reference to the expansion linker, means that if the expansion linker is rotated 180° about the axis defined by the common ligand attachment to the expansion linker, then the positions of the two specificity ligands are exactly swapped. This swapping is such that if the two specificity ligands were identical, the overall conformation of the molecule would be indistinguishable from the conformation before the rotation. As used herein, “approximate C2 symmetry”, when used in reference to the expansion linker, means that if the expansion linker is rotated 180° about the axis defined by the common ligand attachment to the expansion linker, then the positions of the two specificity ligands are approximately swapped. This swapping is such that a given specificity ligand occupies substantially the same position in space as the other specificity ligand did prior to the rotation, within about 5 Å. Approximate C2 symmetry is intended to include perfect C2 symmetry.

As used herein, the term “receptor” refers to a polypeptide that is capable of selectively binding a ligand. Furthermore, the receptor can be a functional fragment or modified form of the entire polypeptide so long as the receptor exhibits selective binding to a ligand. A functional fragment of a receptor is a fragment exhibiting binding to a common ligand and a specificity ligand. Receptors can include, for example, enzymes such as kinases, dehydrogenases, oxidoreductases, GTPases, carboxyl transferases, acyl transferases, decarboxylases, transaminases, racemases, methyl transferases, formyl transferases, and α-ketodecarboxylases. As used herein, the term “enzyme” refers to a molecule that carries out a catalytic reaction by converting a substrate to a product.

Enzymes can also be classified based on Enzyme Commission (EC) nomenclature recommended by the Nomenclature Committee of the International Union of Biochemistry and Molecular Biology (IUBMB)(see, for example, http://www.expasy.ch/sprot/enzyme.html)(which is incorporated herein by reference). For example, oxidoreductases are classified as oxidoreductases acting on the CH—OH group of donors with NAD⁺ or NADP⁺ as an acceptor (EC 1.1.1); oxidoreductases acting on the aldehyde or oxo group of donors with NAD⁺ or NADP⁺ as an acceptor (EC 1.2.1); oxidoreductases acting on the CH—CH group of donors with NAD⁺ or NADP⁺ as an acceptor (EC 1.3.1); oxidoreductases acting on the CH—NH₂ group of donors with NAD+or NADP+as an acceptor (EC 1.4.1); oxidoreductases acting on the CH—NH group of donors with NAD⁺ or NADP⁺ as an acceptor (EC 1.5.1); oxidoreductases acting on NADH or NADPH (EC 1.6); and oxidoreductases acting on NADH or NADPH with NAD⁺ or NADP⁺ as an acceptor (EC 1.6.1).

Additional oxidoreductases include oxidoreductases acting on a sulfur group of donors with NAD⁺ or NADP⁺ as an acceptor (EC 1.8.1); oxidoreductases acting on diphenols and related substances as donors with NAD⁺ or NADP⁺ as an acceptor (EC 1.10.1); oxidoreductases acting on hydrogen as donor with NAD⁺ or NADP⁺ as an acceptor (EC 1.12.1); oxidoreductases acting on paired donors with incorporation of molecular oxygen with NADH or NADPH as one donor and incorporation of two atoms (EC 1.14.12) and with NADH or NADPH as one donor and incorporation of one atom (EC 1.14.13); oxidoreductases oxidizing metal ions with NAD⁺ or NADP⁺ as an acceptor (EC 1.16.1); oxidoreductases acting on —CH₂ groups with NAD⁺ or NADP⁺ as an acceptor (EC 1.17.1); and oxidoreductases acting on reduced ferredoxin as donor, with NAD⁺ or NADP⁺ as an acceptor (EC 1.18.1).

Other enzymes include transferases classified as transferases transferring one-carbon groups (EC 2.1); methyltransferases (EC 2.1.1); hydroxymethyl-, formyl- and related transferases (EC 2.1.2); carboxyl- and carbamoyltransferases (EC 2.1.3); acyltransferases (EC 2.3); and transaminases (EC 2.6.1). Additional enzymes include phosphotransferases such as phosphotransferases transferring phosphorous-containing groups with an alcohol as an acceptor (kinases) (EC 2.7.1); phosphotransferases with a carboxyl group as an acceptor (EC 2.7.2); phosphotransfer with a nitrogenous group as an acceptor (EC 2.7.3); phosphotransferases with a phosphate group as an acceptor (EC 2.7.4); and diphosphotransferases (EC 2.7.6).

Enzymes can also bind coenzymes or cofactors such as nicotinamide adenine dinucleotide (NAD) and nicotinamide adenine dinucleotide phosphate (NADP), thiamine pyrophosphate, flavin adenine dinucleotide (FAD) and flavin mononucleotide (FMN), pyridoxal phosphate, coenzyme A, and tetrahydrofolate or other cofactors or substrates such as ATP, GTP and S-adenosyl methionine (SAM). In addition, enzymes that bind newly identified cofactors or enzymes can also be receptors.

As used herein, the term “receptor family” refers to a group of two or more receptors that share a common, recognizable amino acid motif. A motif can also be known as a pattern, signature or fingerprint. A motif in a related family of receptors occurs because certain amino acid residues are required for the structure, function or activity of the receptor and are therefore conserved between members of the receptor family. The function or activity of a receptor can be enzymatic activity or ligand binding. Methods of identifying related members of a receptor family are well known to those skilled in the art and include sequence alignment algorithms and identification of conserved patterns or motifs in a group of polypeptides, which are described in more detail below. Members of a receptor family also bind a natural common ligand, which can be verified in a binding assay after the receptor is cloned and expressed.

As used herein, the term “conserved site” refers to the amino acid residues sufficient for activity or function of the receptor that are accessible to binding of a natural common ligand. A conserved site is common to members of a receptor family. For example, the amino acid residues sufficient for activity or function of a receptor that is an enzyme can be amino acid residues in a substrate binding site of the enzyme. Also, the conserved site in an enzyme that binds a cofactor or coenzyme can be amino acid residues that bind the cofactor or coenzyme.

As used herein, the term “population” refers to a group of two or more different molecules. A population can be as large as the number of individual molecules currently available to the user or able to be made by one skilled in the art. A population can be as small as two molecules and as large as 10¹⁰ molecules. Generally, a population will contain two or more, three or more, five or more, ten or more, or twenty of more different molecules. A population can also contain tens or hundreds of different molecules or even thousands of different molecules. For example, a population can contain about 100 to 100,000 different molecules or more, and particularly about 10,000 different molecules. A population of bi-ligands is derived, for example, by chemical synthesis and is substantially free of naturally occurring substances.

As used herein, a “library” is comprised of a population of different molecules. The library is chemically synthesized and contains primarily the components generated during the synthesis.

As used herein, the term “specificity” refers to the ability of a ligand to differentially bind to one receptor over another in the same receptor family. The differential binding of a particular ligand to a receptor is measurably higher than the binding of the ligand to at least one other receptor in the same receptor family. Specificity can also be exhibited over two or more other members of the receptor family, five or more other members of the receptor family or even ten or more other members of the receptor family.

As used herein, the term “specificity site” refers to a site on a receptor that imparts molecular properties that distinguish the receptor from other receptors in the same receptor family. The specificity site on a receptor provides the binding site for a ligand exhibiting specificity for a receptor. For example, if the receptor is an enzyme, the specificity site can be a substrate binding site that distinguishes two members of a receptor family that exhibit substrate specificity. A substrate specificity site can be exploited as a potential binding site for the identification of a ligand that has specificity for one receptor over another member of the same receptor family.

The invention provides methods A for generating a library of bi-ligands. The methods consist of (a) determining a common ligand to a conserved site in a receptor family; (b) attaching an expansion linker to said common ligand, wherein said expansion linker has sufficient length and orientation to direct a second ligand to a specificity site of a receptor in said receptor family, to form a module; and (c) generating a population of bi-ligands comprising a plurality of identical modules attached to variable second ligands.

The invention also provides methods further comprising (d) screening said population of bi-ligands for binding to a receptor in said receptor family; and (e) identifying a bi-ligand that binds to and has specificity for said receptor.

The invention additionally provides methods for identifying a population of bi-ligands to receptors in a receptor family. The methods consist of (a) determining a common ligand to a conserved site in the receptor family; (b) attaching an expansion linker to said common ligand, wherein said expansion linker has sufficient length and orientation to direct a second ligand to a specificity site of a receptor in said receptor family, to form a module; and (c) generating a population of bi-ligands, wherein said bi-ligand comprises said module and a second ligand linked by said expansion linker.

The invention additionally provides methods further comprising: (d) screening said population of bi-ligands for binding to a receptor in said receptor family; (e) identifying a bi-ligand that binds to and has specificity for said receptor; and (f) repeating steps (d) and (e) to identify a bi-ligand that binds to and has specificity for a second receptor in said receptor family.

A common ligand attached to an expansion linker is a “module” on which to build a population of bi-ligands. Attachment of various second ligands to generate a population of bi-ligands and screening for a high affinity, high specificity ligand also enhances the probability of identifying such a ligand due to the synergistic effect of tethering two ligands that bind to a receptor. Additionally, the use of a module comprising a common ligand attached to an expansion linker means that once a population of bi-ligands has been generated, the same population can be used to screen for high affinity, high specificity ligands for other members of the same receptor family. An additional advantage is that, when the target receptor is a newly identified or uncharacterized gene product, it is not necessary to know or determine a natural ligand for the receptor because specificity is generated by screening an oriented population of bi-ligands. Thus, this modular, oriented approach provides a more efficient method to identify high affinity, high specificity ligands to a target receptor.

Initially, a target disease is identified for the development of a ligand useful as a therapeutic agent. After identification of a target disease, a cell or organism responsible for the target disease is selected, and a receptor family expressed in the organism is identified for targeting of a ligand. For example, a pathogen can be selected as the target organism to develop drugs effective in combating a disease caused by that pathogen. Any pathogen can be selected as a target organism. Examples of pathogens include, for example, bacteria, fungi or protozoa. In addition, a target cell such as a cancer cell can be selected to identify drugs effective for treating cancer. Examples of such target cells include, for example, breast cancer, prostate cancer, and ovarian cancer cells as well as leukemia, lymphomas, melanomas, sarcomas and gliomas.

In one embodiment, a bacterium is selected as a target organism. Pathogenic bacteria useful as target organisms include Staphylococcus, Mycobacteria, Mycoplasma, Streptococcus, Haemophilus, Neisseria, Bacillus, Clostridium, Corynebacteria, Salmonella, Shigella, Vibrio, Campylobacter, Helicobacter, Pseudomonas, Legionella, Bordetella, Bacteriodes, Fusobacterium, Yersinia, Actinomyces, Brucella, Borrelia, Rickettsia, Ehrlichia, Coxiella, Chlamydia, and Treponema. Pathogenic strains of Escherichia coli can also be target organisms.

Ligands targeted to receptors in these pathogenic bacteria are useful for treating a variety of diseases including bacteremia, sepsis, nosocomial infections, pneumonia, pharyngitis, scarlet fever, necrotizing fasciitis, abscesses, cellulitis, rheumatic fever, endocarditis, toxic shock syndrome, osteomyelitis, tuberculosis, leprosy, meningitis, pertussis, food poisoning, enteritis, enterocolitis, diarrhea, gastroenteritis, shigellosis, dysentery, botulism, tetanus, anthrax, diphtheria, typhoid fever, cholera, actinomycosis, Legionnaire's disease, gangrene, brucellosis, lyme disease, typhus, spotted fever, Q fever, urethritis, vaginitis, gonorrhea and syphilis.

For example, Staphylococcus aureus is a major cause of nosocomial infections and has become increasingly resistant to a variety of antibiotics over recent years. Similarly, Mycobacteria tuberculosis has become increasingly resistant to multiple antibiotics in recent years. M. tuberculosis infects almost one third of the world population, with active tuberculosis found in almost 10 million people worldwide and in AIDS patients as a common opportunistic infection. Streptomyces has also become increasingly resistant to antibiotics over recent years. Therefore, these pathogenic bacteria with known resistance are particularly desirable as target organisms for identifying ligands that bind target receptors.

In another embodiment, target organisms are selected from yeast and fungi. Pathogenic yeast and fungi useful as target organisms include Aspergillus, Mucor, Rhizopus, Candida, Cryptococcus, Blastomyces, Coccidioides, Histoplasma, Paracoccidioides, Sporothrix, and Pneumocystis. Ligands targeted to receptors in these pathogenic yeast and fungi are useful for treating a variety of diseases including aspergillosis, zygomycosis, candidiasis, cryptococcoses, blastomycosis, coccidioidomycosis, histoplasmosis, paracoccidioidomycosis, sporotrichosis, and pneuomocystis pneumonia.

In still another embodiment, target organisms are selected from protozoa. Pathogenic protozoa useful as target organisms include Plasmodium, Trypanosoma, Leishmania, Toxoplasma, Cryptosporidium, Giardia, and Entamoeba. Ligands targeted to receptors in these pathogenic protozoa are useful for treating a variety of diseases including malaria, sleeping sickness, Chagas' disease, leishmaniasis, toxoplasmosis, cryptosporidiosis, giardiasis, and amebiasis.

After identifying a target organism or cell, all available genetic information about the organism or cell is reviewed. If sufficient genetic information is available, a target receptor family is chosen. Sufficient genetic information is available for an organism or cell if there are at least two members of a receptor family for which genetic information is available. The entire sequence of the members of the receptor family need not be known, only sufficient sequence information to determine that the receptors are in the same receptor family.

Methods for determining that two receptors are in the same family are well known in the art. For example, one method for determining if two receptors are related is BLAST, Basic Local Alignment Search Tool, available on the National Center for Biotechnology Information web page (http://www.ncbi.nlm.gov/BLAST/)(which is incorporated herein by reference). BLAST is a set of similarity search programs designed to examine all available sequence databases and can function to search for similarities in protein or nucleotide sequences. A BLAST search provides search scores that have a well-defined statistical interpretation. Furthermore, BLAST uses a heuristic algorithm that seeks local alignments and is therefore able to detect relationships among sequences which share only isolated regions of similarity (Altschul et al., J. Mol. Biol. 215:403-410 (1990), which is incorporated herein by reference).

In addition to the originally described BLAST (Altschul et al., supra, 1990), modifications to the algorithm have been made (Altschul et al., Nucleic Acids Res. 25:3389-3402 (1997), which is incorporated herein by reference). One modification is Gapped BLAST, which allows gaps, either insertions or deletions, to be introduced into alignments. Allowing gaps in alignments tends to reflect biologic relationships more closely. A second modification is PSI-BLAST, which is a sensitive way to search for sequence homologs. PSI-BLAST performs an initial Gapped BLAST search and uses information from any significant alignments to construct a position-specific score matrix, which replaces the query sequence for the next round of database searching. A PSI-BLAST search is often more sensitive to weak but biologically relevant sequence similarities.

A second resource for identifying members of a receptor family is PROSITE, available at ExPASy (http://www.expasy.ch/sprot/prosite.html)(which is incorporated herein by reference). PROSITE is a method of determining the function of uncharacterized proteins translated from genomic or cDNA sequences (Bairoch et al., Nucleic Acids Res. 25:217-221 (1997), which is incorporated herein by reference). PROSITE consists of a database of biologically significant sites and patterns that can be used to identify which known family of proteins, if any, the new sequence belongs. In some cases, the sequence of an unknown protein is too distantly related to any protein of known structure to detect its resemblance by overall sequence alignment. However, related proteins can be identified by the occurrence in its sequence of a particular cluster of amino acid residues, which can be called a pattern, motif, signature or fingerprint. PROSITE uses a computer algorithm to search for motifs that identify proteins as family members. PROSITE also maintains a compilation of previously identified motifs, which can be used to determine if a newly identified protein is a member of a known protein family.

A third resource for identifying members of a receptor family is Structural Classification of Proteins (SCOP) available at SCOP (http://scop.mrc-lmb.cam.ac.uk/scop/)(which is incorporated herein by reference). Similar to PROSITE, SCOP maintains a compilation of previously determined protein motifs for comparison and determination of related proteins (Murzin et al., J. Mol. Biol. 247:536-540 (1995), which is incorporated herein by reference).

Additional resources for identifying motifs of a receptor family are shown in Table 1. The websites sited therein are incorporated by reference.

TABLE 1
Databases for Identifying Receptor Family Motifs

| | WEBSITES |
|---|---|
| SEARCHABLE MOTIF AND | |
| PATTERN DATABASES | |
| PROSITE | http://expasy.hcuge.ch/sprot/prosite.html |
| BLOCKS | http://www.blocks.fhcrc.org/blocks_search.html |
| PRINTS | http://www.biochem.uc1.ac.uk/bsm/dbbrowser/PRIN |
| | TS/PRINTS.html |
| PIMA | http://dot.imgen.bcm.tmc.edu:9331/seq- |
| | search/protein- search.html |
| PRODOM | http://protein.toulouse.inra.fr/prodom.html |
| MOTIF AND PROFILE SEARCHES | |
| REGULAR EXPRESSION SEARCH | http://www.ibc.wustl.edu/fpat/ |
| PROFILESEARCH | http://www.seqnet.dl.ac.uk/hhg/PROFILESE.h |
| | tml |
| PATSCAN | http://www |
| | c.mcs.anl.gov/home/overbeek/PatScan/HTML/patsca |
| | n.html |
| PATTERNFIND | http://ulrec3.unil.ch/software/PATFND- |
| | mailform.html |
| PROFILE | http://lenti.med.umn.edu/MolBio_man/chp- |
| | 10.html#HDR1 |
| PMOTIF | http://alces.med.umn.edu/pmotif.html |
| HMMER | http://genome.wustl.edu/eddy/HMMER/ |
| WWW AND FTP SERVERS FOR | |
| SINGLE SEQUENCE EXHAUSTIVE | |
| DATABASE SEARCHES | |
| BLAST | http://www.ncbi.nlm.nih.gov/BLAST/ |
| BLITZ | http://www.ebi.ac.uk/searches/blitz_input.html |
| FASTA | http://www.genome.ad.jp/ideas/fasta/fasta_genes.html |
| FTP ADDRESSES FOR MOTIF | |
| AND PROFILE SEARCH PROGRAMS | |
| BARTON'S FLEXIBLE PATTERNS | ftp://geoff.biop.ox.ac.uk/ |
| PROPAT | ftp://ftp.mdc-berlin.de/ |
| SOM | ftp://ftp.mdc-berlin.de/pub/neural |
| SEARCHWISE | ftp://sable.ox.ac.uk/pub/users |
| PROFILE | ftp://ftp.ebi.ac.uk/pub/software/unix/ |
| TPROFILESEARCH | ftp://ftp.ebi.ac.uk/pub/softare/vax/egcg |
| CAP | ftp://ncbi.nlm.nih.gov/pub/koonin/cap |

Conserved amino acids are evolutionarily conserved to carry out a common function. For example, the Rossman fold is a tertiary structural motif that includes GXGXXG and is present in enzymes that bind nucleotides (Brandon and Tooze, in Introduction to Protein Structure, Garland Publishing, New York (1991), which is incorporated herein by reference). Enzymes that bind nucleotides such as NAD, NADP, FAD, ATP, ADP, AMP and FMN contain the Rossman fold sequence motif (Creighton, Proteins: Structures and Molecular Principles, p.368, W.H. Freeman, New York (1984), which is incorporated herein by reference). Additional conserved residues as well as different protein structures distinguish receptor families that bind, for example, NAD from those that bind, for example, ATP.

An example of a recognizable protein motif or fingerprint is found in dinucleotide binding proteins such as dehydrogenases (Rossman et al., in The Enzymes Vol 11, Part A, 3rd ed., Boyer, ed., pp. 61-102, Academic Press, New York (1975); Wierenga et al., J. Mol. Biol. 187:101-107 (1986); and Ballamacina, FASEB J. 10:1257-1269 (1996), each of which is incorporated herein by reference). The fingerprint region comprises a phosphate binding consensus sequence GXGXXG, a hydrophobic core of six small hydrophobic residues, a conserved, negatively charged residue that binds to the ribose 2′ hydroxyl of adenine and a conserved positively charged residue (Bellamacina, supra).

Protein kinases also have recognizable motifs conserved among all known protein kinases (Hanks and Quinn, Methods Enzymol. 200:28-62 (1991), which is incorporated herein by reference). Eight invariant amino acid residues are conserved throughout the protein kinase family, including a conserved GXGXXG motif similar to that seen in dinucleotide binding proteins. A crystallographic molecular model of cyclic AMP-dependent protein kinase as well as other protein kinases showed that these conserved residues are nearly all associated with essential, conserved functions such as ATP binding and catalysis (Knighton et al., Science 253:407-414 (1991); and Knighton et al., Science 253:414-420 (1991)). Thus, conserved amino acid residues, which are common to members of a protein family, are recognizable as a motif critical for the structure, function or activity of a protein.

Pyridoxal binding receptors also have recognizable motifs. One motif is GXGGXXXG, a second motif is KXEX₆SXKX₅₋₆M, and a third motif is PXNPTG (Suyama et al., Protein Engineering 8:1075-1080 (1995), which is incorporated herein by reference).

A receptor family is selected based on a conserved and recognizable motif such as those described above and in the public databases. Once a receptor family has been identified, a determination is made as to whether the receptor family is useful for identifying ligands as potential therapeutic agents. This is done by determining if the receptor family has a natural common ligand that binds to at least two members of the receptor family, and preferably to several or most members of the receptor family.

In many cases, an identified receptor family will have a natural common ligand that is already known. For example, it is known that dehydrogenases bind to dinucleotides such as NAD or NADP. Therefore, NAD or NADP are natural common ligands to a number of dehydrogenase family members. Similarly, all kinases bind ATP, which is therefore a natural common ligand to kinases. Other natural common ligands of a receptor family can be the coenzymes and cofactors described above.

After a receptor family has been determined, at least two receptors in the receptor family are selected as drug targets for identifying ligands useful as therapeutic agents. The criteria for selection of receptor family members depends on the needs of the user. For example, if the receptor family is from a pathogenic organism, the receptor family members selected can be those most divergent from the organism to be treated with the therapeutic agent. If the organism to be treated is a mammal such as human, then the receptor family members from the pathogenic organism are compared to known mammalian or human members of the receptor family. Methods of comparing protein sequences are well known in the art and include BLAST as described above. Those receptors that are most distantly related to human can then be selected for identifying ligands as therapeutic agents since it is easier to identify ligands having higher specificity for the pathogenic organism if the target receptor is more divergent.

If the receptor family is from a target cell such as a cancer cell, target receptors in a receptor family can be selected based on the criteria that the target receptor is more highly expressed or is more active in a cancer cell. A ligand targeted to such a receptor will be more likely to affect the target cancer cell rather than other non-cancerous cells in the organism.

After a target receptor is selected, the selected receptor or functional fragment thereof is cloned and expressed. Methods for cloning a gene encoding a receptor target are well known to those skilled in the art and include, for example, polymerase chain reaction (PCR) and other molecular biology techniques (Dieffenbach and Dveksler, eds., PCR Primer: A Laboratory Manual, Cold Spring Harbor Laboratory Press, Plainview, N.Y. (1995); Sambrook et al., Molecular Cloning: A Laboratory Manual, 2nd ed., Cold Spring Harbor Laboratory Press, Plainview, N.Y. (1989); Ausubel et al., Current Protocols in Molecular Biology, Vols. 1-3, John Wiley & Sons (1998), each of which is incorporated herein by reference). The target receptor gene is cloned into an appropriate expression vector for expression in an organism such as bacteria, insect cells, yeast or mammalian cells. Target receptors are preferably expressed in organisms that can be grown in defined media for the NMR experiments described below.

If desired, the target receptor can be expressed as a fusion protein with a tag that facilitates purification of the receptor. Because nuclear magnetic resonance (NMR) experiments will be performed on the target receptor, the tag is preferably a small polypeptide. For example, the target receptor can be expressed as a fusion with a poly-His tag, which can be purified by metal chelate chromatography. Other useful affinity purification tags which can be expressed as fusions with the target receptor and affinity purified include glutathione S transferase (GST) and myc, which are engineered with specific protease cleavage sites for cleavage and removal of the affinity tag following affinity chromatography.

The target receptor can be validated as a representative member of a receptor family. In some cases, the target receptor is well characterized with respect to its binding properties to a natural common ligand of a receptor family. However, if the target receptor is encoded by a new, uncharacterized gene, the expressed target receptor can be tested to confirm that the natural common ligand of the selected receptor family does bind to the target receptor. Other natural common ligands of distantly related receptor families, for example other nucleotide binding receptors, can also be tested for binding to the target receptor.

The target receptor can be further validated as a useful therapeutic target by determining if the selected target receptor is known to be required for normal growth, viability or infectivity of the target organism or cell. If it is unknown whether the target receptor is required for normal growth, viability, or infectivity the target receptor can be specifically inactivated by gene knockout to determine if the receptor performs a critical function required for survival or infectivity of the organism or cell. Such a receptor providing a critical function is a good target for developing therapeutic agents.

Methods for disrupting a gene to generate a knockout are well known in the art (Ausubel et al., Current Protocols in Molecular Biology, Vols 1-3, John Wiley & Sons (1998), which is incorporated herein by reference). For example, transposable elements can be used to knockout a gene and test for the effect of the knockout on cell growth, viability or infectivity (Benson and Goldman, J. Bacteriol. 174:167301681 (1992); Hughes and Roth, Genetics 119:9-12 (1988); and Elliot and Roth, Mol. Gen. Genet. 213:332-338 (1988), each of which is incorporated herein by reference). Methods for gene knockouts in protozoa have also been previously described (Wang, Parasitology 114:531-544 (1997); and Li et al, Mol. Biochem. Parasitol. 78:227-236 (1996), each of which is incorporated herein by reference).

A bi-ligand is identified by initially determining a common ligand that binds to at least two target receptors in a receptor family. The use of a common ligand is advantageous for rapidly identifying a high affinity ligand that is a potential therapeutic agent. It is well known that the combination of two ligands into a single molecule that allows both ligands to simultaneously bind to a receptor provides synergistically higher affinity than either ligand alone (Dempsey and Snell, Biochemistry 2:1414-1419 (1963); and Radzicka and Wolfenden, Methods Enzymol. 249:284-303 (1995), each of which is incorporated herein by reference). By starting with a common ligand and attaching an expansion linker to which a variety of a second specificity ligands can be attached, a high affinity bi-ligand is identified more rapidly than screening a pool of compounds, as when screening a randomly generated combinatorial library. Furthermore, the use of a common ligand, when attached to other specificity ligands, also allows the identification of multiple ligands capable of binding to multiple members of a receptor family. Thus, the common ligand and expansion linker act together as a re-usable module in the generation of multiple bi-ligands that can inhibit activity or function of other receptors in a given receptor family.

As described above, in some cases, a common ligand to a receptor family is already known. For example, NAD is a natural common ligand for dehydrogenases, and ATP is a natural common ligand for kinases. In addition to naturally occurring substrates and cofactors, analogs of these substrates and cofactors that bind to a conserved site are also often known. However, natural common ligands such as the coenzymes and cofactors described above and known derivatives thereof often have limitations regarding their usefulness as a starting compound. Substrates and cofactors often undergo a chemical reaction, for example, transfer of a group to another substrate or reduction or oxidation during the enzymatic reaction. However, it is desirable that a ligand to be used as a drug is not metalizable.

Additionally, the common ligand is used as a platform to attach specificity ligands capable of binding to a specificity site of a receptor. This requires that the common ligand and specificity ligand be oriented for optimized binding to the conserved site and specificity site. However, the position on a natural common ligand that is oriented towards a specificity site is not always readily derivatizable for attaching a chemical group. Finally, some substrates or cofactors are highly charged, often making them less able to cross the membrane to target a receptor inside the cell. Therefore, it is often desirable to identify additional common ligands that are useful for generating bi-ligands.

Methods of screening for a common ligand are well known in the art. For example, a receptor can be incubated in the presence of a known ligand and one or more potential common ligands. In some cases, the natural common ligand has an intrinsic property that is useful for detecting whether the natural common ligand is bound. For example, the natural common ligand for dehydrogenases, NAD, has intrinsic fluorescence. Therefore, increased fluorescence in the presence of potential common ligands due to displacement of NAD can be used to detect competition for binding of NAD to a target NAD binding receptor (Li and Lin, Eur. J. Biochem. 235:180-186 (1996); and Ambroziak and Pietruszko, Biochemistry 28:5367-5373 (1989), each of which is incorporated herein by reference).

In other cases, when the natural common ligand does not have an intrinsic property useful for detecting ligand binding, the known ligand can be labeled with a detectable moiety. For example, the natural common ligand for kinases, ATP, can be radiolabeled with ³²P, and the displacement of radioactive ATP from an ATP binding receptor in the presence of potential common ligands can be used to detect additional common ligands. Any detectable moiety, for example a radioactive or fluorescent label, can be added to the known ligand so long as the labeled known ligand can bind to a receptor having a conserved site.

The pool of potential common ligands screened for competitive binding with a natural common ligand can be a broad range of compounds of various structures. However, the pool of potential ligands can also be focused on compounds that are more likely to bind to a conserved site in a receptor. For example, a pool of candidate common ligands can be chosen based on structural similarities to the natural common ligand or a mimic thereof. The pool of potential common ligands is a group of analogs and mimetics of the natural common ligand.

One approach to identify common ligands is to perform high throughput screening on a large pool of commercially available molecules. Common ligands identified by the methods described above can be further characterized by NMR as described below.

Another approach is to use the three-dimensional structure of a natural common ligand and search commercially available databases of commercially available molecules such as the Available Chemicals Directory (MDL Information Systems, Inc.; San Leandro Calif.) to identify potential common ligands having similar shape or electrochemical properties of the natural common ligand. Methods for identifying molecules having similar structure are well known in the art and are commercially available (Doucet and Weber, in Computer-Aided Molecular Design: Theory and Applications, Academic Press, San Diego Calif. (1996), which is incorporated herein by reference; software is available from Molecular Simulations, Inc., San Diego Calif.). Furthermore, if structural information is available for the conserved site in the receptor, particularly with a known ligand bound, compounds that fit the conserved site can be identified through computational methods (Blundell, Nature 384 Supp: 23-26 (1996), which is incorporated herein by reference).

Once a pool of potential common ligands is selected, the pool is screened, for example, by competition with a natural common ligand, to determine at least one common ligand that binds to a conserved site in a target receptor. The common ligands identified by the screen are then further characterized with respect to affinity of the common ligands to the target receptor. Generally it is desirable to identify a common ligand that is not a high affinity ligand. Since the common ligand binds to multiple members of a receptor family, a high affinity common ligand would likely bind to other members of a receptor family in addition to the target receptor. It is therefore desirable to identify common ligands having modest affinity, preferably at or below the affinity of the natural common ligand that binds to the same conserved site. Such a common ligand having modest affinity is then used as a starting compound for identifying a bi-ligand. Generally, modest affinity ligands will have affinity for a receptor of about 10-2 to 10⁻⁷ M, particularly about 10⁻³ to 10⁻⁶ M.

When multiple common ligands are initially determined having desired characteristics such as modest affinity, at least one of the common ligands is selected as a starting compound for identifying a bi-ligand. Because the bi-ligands will bind to both a common site and a specificity site on a receptor, it is desirable to determine if a common ligand binds to the conserved site near the specificity site of a receptor. This further assures that an expansion linker, when attached to the common ligand, will be in an optimized position and have optimized characteristics for attaching a specificity ligand.

For example, if a common ligand binds to a region of the conserved site distal to the specificity site, the generation of a bi-ligand requires a longer expansion linker than if it binds to a region proximal to the specificity site. A longer expansion linker generates a bi-ligand that is less likely to be a high affinity ligand due to unfavorable entropic characteristics that allows the common ligand relative to the specificity ligand to assume a larger number of conformations than when the expansion linker is smaller. A smaller expansion linker also makes the bi-ligand smaller, which is generally a desirable characteristic for an effective drug.

One method useful for determining a ligand that binds to a region of a conserved site in closest proximity to a specificity site is NMR spectroscopy. Whereas the usual application of NMR for characterizing a binding site for a common ligand requires the time consuming process of assigning all protons and heteronuclei and is usually limited to proteins of size less than 30 kDa, the present invention provides methods to obtain limited structural information sufficient to determine that a common ligand occupies a region of a conserved site in proximity to a specificity site. The NMR spectroscopy data can also be used to determine where it would be best to attach the expansion linker onto the common ligand such that the specificity ligand is in a position that is optimized for binding to the specificity site.

To perform the NMR experiments, a target receptor is expressed in bacteria, yeast or other suitable organisms that can be grown on defined media. Receptors up to about 70 kDa can be readily used for NMR spectroscopy experiments if they are deuterated. The organism is grown in the presence of D₂O in place of water, as well as a nitrogen source with ¹⁵N, for example, salts of ¹⁵NH₄⁺ such as (¹⁵NH₄)₂SO₄ or ¹⁵NH₄Cl. The sole carbon source is acetate or glucose if complete deuteration on carbon is desired. If pyruvate is used as the sole carbon source, there will be protons only on the methyl groups of Ala, Val, Leu and Ile (Kay, Biochem. Cell Biol. 75:1-15 (1997), which is incorporated herein by reference). These and other related methods for isotopically labeling proteins have been described previously (Laroche, et al., Biotechnology 12:1119-1124 (1994); LeMaster Methods Enzymol. 177:23-43 (1989); Muchmore et al., Methods Enzymol. 177:44-73 (1989); Reilly and Fairbrother, J. Biomolecular NMR 4:459-462 (1994); Ventors et al., J. Biomol. NMR 5:339-344 (1995); and Yamazaki et al., J. Am. Chem. Soc. 116:11655-11666 (1994), each of which is incorporated herein by reference). Since all other protons are replaced with deuterons, NMR line widths are narrow enough that heteronuclear single quantum coherence (HSQC) spectroscopy data can be gathered on the target receptor.

The deuterated ¹⁵N-labeled receptor is purified, for example, by affinity chromatography, and incubated in H₂O to replace deuterons on the amides with protons. For NMR experiments, two-dimensional ¹H—¹⁵N HSQC spectra of the receptor in the presence and absence of a common ligand are obtained that is sufficient to identify protein protons based on their binding function only. It is therefore not necessary to obtain a complete NMR structural model of the target receptor but merely to obtain sufficient information to determine which amino acid residues in the conserved site contact the common ligand or are close to the common ligand. The use of NMR spectroscopy to identify amino acids involved in ligand interactions has been described previously (Davis et al., J. Biomolecular NMR 10:21-27 (1997); Hrovat et al., J. Biomolecular NMR 10:53-62 (1997); and Sem et al., J. Biol. Chem. 272:18038-18043 (1997), each of which is incorporated herein by reference).

In order to define which NMR cross peaks belong to amino acid residues in the part of the conserved site proximal to a specificity site, NMR experiments are performed with the target receptor in the presence of a natural common ligand and in the presence of a modified version of the natural common ligand that provides information on the orientation of the specificity site of the receptor relative to the conserved site. In the case where the natural common ligand is a substrate, for example, additional NMR spectra in the presence of a modified natural common ligand such as the product can be performed to determine difference spectra between the binding of substrate and product. Since the difference between the substrate and product is necessarily in the portion of the common site oriented towards the specificity site, where a specific substrate binds, the difference spectra therefore indicate which NMR cross peaks belong to amino acid residues that are proximal to the specificity site. The method does not require assigning these cross peaks to specific amino acid residues.

For example, in the case of a kinase receptor, NMR experiments can be performed in the presence of a kinase receptor and ATP or ADP. Since the transfer of phosphate from ATP to substrate is the reaction catalyzed by kinases, the γ-phosphate of ATP is necessarily proximal to the specificity site that binds the substrate of the kinase. In another example, the ATP analog could be chromium ATP since the phosphates of ATP are oriented toward the specificity site. Chromium ATP or chromium ADP can be prepared as previously described (Cleland, Methods Enzymol. 87:159-179 (1982), which is incorporated herein by reference). In still another example, in the case of a NAD binding protein such as a dehydrogenase, the NAD molecule can be modified, for example, by separately binding adenine mononucleotide or nicotinamide mononucleotide. Since nicotinamide is the group that accepts electrons during a dehydrogenase reaction, the nicotinamide group is necessarily more proximal to the specificity site than the adenine group.

By comparing 2D ¹H—¹⁵N HSQC spectra for natural common ligands that have been modified in the manner described above, NMR cross peaks that change their ppm (part per million) values most significantly are identified as belonging to amino acid residues close in space to where the natural common ligand has been altered. Thus, using a modified version of a natural common ligand along with the natural common ligand in an NMR experiment identifies the portion of the common site proximal to the specificity site.

After NMR cross peaks belonging to amino acid residues of the conserved site are identified as proximal to the specificity site, the common ligands that compete for binding of the natural common ligand described above are tested for their ability to bind to the same residues in the conserved site that are proximal to the specificity site. This is carried out by performing similar NMR experiments, which are based on the observation of perturbed chemical shifts in the 2D ¹H—¹⁵N HSQC spectra for the identified cross peaks, such as those described above and identifying those common ligands that bind to the amino acid residues of the conserved site that are proximal to the specificity site. In this way, common ligands that bind to a conserved site and are proximal to a specificity site are determined.

The portion of the common ligand closest to the amino acids proximal to the specificity site are determined by 3D HSQC-NOESY (nuclear Overhauser effect spectroscopy) experiments on the common ligand/receptor complex. The NMR experiments measure nuclear Overhauser effects (NOEs) between the receptor proton cross peaks, identified based on their proximity to the specificity site, and the common ligand. An NOE is only observed between two protons that are within 5 Å of each other. Therefore, NOE measurements between these receptor protons and the common ligand indicate which protons on the common ligand are within 5 Å of the protons on the receptor that are proximal to the specificity site. This is accomplished without having to do complete proton assignments. The NOEs between cross peaks on the receptor that were identified as proximal to the specificity site and protons on the common ligand are determined.

The NMR experiments also provide useful information regarding the orientation of the common ligand. The most efficient way to identify a bi-ligand that has high affinity and specificity for a receptor is to attach an expansion linker to a common ligand in an orientation that is optimized for attaching a specificity linker such that both the common ligand and specificity ligand bind to their respective sites on the receptor. The NOE measurements between receptor and common ligand not only provide information on which cross peaks belong to amino acid residues of the receptor proximal to the bound ligand but also give information on which chemical group protons of the common ligand are proximal to the binding amino acid residues in the receptor in the portion of the common site proximal to the specificity site, since those proton cross peaks were identified in the difference 2D HSQC experiments.

Information on the interactions between receptor and ligand can be obtained using heteronuclear NMR experiments, including 2D HSQC, 3D HSQC-NOESY and 3D NOESY-HSQC (Cavanagh et al., in Protein NMR Spectroscopy: Principles and Practice, Academic Press, San Diego (1996), which is incorporated herein by reference). The above method describes how to identify the chemical groups of the ligand that bind to the amino acid residues in the conserved site proximal to the specificity site. The chemical group or groups of the common ligand that bind to the amino acid residues of the conserved site proximal to the specificity site are preferred sites for attaching an expansion linker to orient a specificity ligand to a specificity site. The method is also advantageous because it is faster than the traditional NMR method that requires the complete assignment of protons and heteronuclei.

The specificity ligand is attached to the common ligand by an expansion linker, which is attached to the common ligand at a position so that the expansion linker is oriented towards the specificity site. An expansion linker has sufficient length and orientation to direct a specificity ligand to a specificity site. The expansion linker is designed to have at least two positions for attaching at least two ligands. One of the positions is used to attach the expansion linker to a common ligand. The other position is used for attaching a specificity ligand.

For some bi-ligands, the expansion linker can be any molecule that provides sufficient length and orientation for directing a second ligand to a specificity site of a receptor. Therefore, any chemical group that provides the appropriate orientation and positioning of the common ligand relative to the specificity ligand for optimized binding to their respective sites on the receptor can be used as an expansion linker.

For some bi-ligands, it is desirable to use an expansion linker that has three positions for attaching ligands, one for attaching a common ligand and two additional positions for attaching one or two specificity ligands. For such bi-ligands, the expansion linker is preferably a molecule that can provide approximate C2 symmetry. The symmetrical feature of the expansion linker is particularly useful for generating bi-target ligands since the approximate C2 symmetry allows the common ligand and one of the specificity ligands to bind in a bi-valent manner to either of two receptors in the same receptor family. The symmetry generated after attachment of specificity ligands to an expansion linker is determined by the specific positions on the expansion linker to which the specificity ligands are attached. Therefore, the same expansion linker can be used to generate perfect C2 symmetry or approximate C2 symmetry. This concept is depicted in FIG. 3 for a representative expansion linker.

Expansion linkers that are useful for generating bi-ligands that do not have C2 symmetry include, for example, substituted phosgene, urea, furane and salicylic acid. However, any chemical group with two reactive sites that can be used to position a common ligand and a specificity ligand in an optimized position for binding to their respective sites can be used as an expansion linker when C2 symmetry is not required.

Expansion linkers that are useful for providing approximate C2 symmetry include, for example, substituted piperidine, pyrrolidine, morpholine, 2,4 di-bromobenzoate, 2-hydroxy-1,4-naphthoquinone, tartaric acid, indole, isoindazole, 1,4-benzisoxazine, phenanthrene, carbazole, purine, pyrazole and 1,2,4-triazole. However, any chemical group with three reactive sites, two of which allow symmetrical attachment of specificity ligands, can be used as an expansion linker when C2 symmetry or approximate C2 symmetry is required in a bi-ligand or bi-target ligand.

Another group of expansion linkers includes molecules containing phosphorous. These phosphorus-containing molecules include, for example, substituted phosphate esters, phosphonates, phosphoramidates and phosphorothioates. The chemistry of substitution of phosphates is well known to those skilled in the art (Emsley and Hall, The Chemistry of Phosphorous: Environmental, Organic, Inorganic and Spectroscopic Aspects, Harper & Row, New York (1976); Buchwald et al., Methods Enzymol. 87:279-301 (1982); Frey et al., Methods Enzymol. 87:213-235 (1982); Khan and Kirby, J. Chem. Soc. B:1172-1182 (1970), each of which is incorporated herein by reference). A related category of expansion linkers includes phosphinic acids, phosphonamidates and phosphonates, which can function as transition state analogs for cleavage of peptide bonds and esters as described previously (Alexander et al., J. Am. Chem. Soc. 112:933-937 (1990), which is incorporated herein by reference). The phosphorous-containing molecules useful as expansion linkers can have various oxidation states, both higher and lower, which have been well characterized by NMR spectroscopy (Mark et al., Progress in NMR Spectroscopy 16:227-489 (1983), which is incorporated herein by reference).

The reactive groups on the expansion linker and the ligands to be attached should be reactive with each other to generate a covalent attachment of the ligand to the expansion linker in the orientation for binding of the common ligand and specificity ligand to their respective binding sites on the receptor. A preferred reaction is that of a nucleophile reacting with an electrophile. Many of the above described expansion linkers have electrophilic groups available for attaching ligands. Electrophilic groups useful for attaching ligands include electrophiles such as carbonyls, alkenes, activated esters, acids and alkyl and aryl halides.

The expansion linkers having electrophilic groups are preferably attached to common ligands having nucleophilic groups positioned for attachment of the ligands in an orientation for binding of the common ligand and specificity ligand. Desirable common ligands can have, for example, alcohols, amines, or mercaptans. However, if a common ligand is identified that does not have appropriate reactive groups for attaching a ligand in a desired orientation to the expansion linker or if the ligand cannot be modified to generate an appropriate reactive group in a desired position, an additional screen is performed, as described above, to identify a common ligand having desired binding characteristics as well as a chemical group in the proper position to achieve a desired orientation of ligands after covalently linking a ligand to the expansion linker.

Reactive positions on the expansion linker can be modified, for example, with hydroxyl, amino or mercapto groups. Therefore, ligands containing reactive hydroxyl, amino or mercapto groups positioned so that, after attaching a specificity ligand, the expansion linker orients the common ligand and specificity ligand to their respective sites on the receptor can be reacted with the expansion linkers described above.

After the expansion linker is attached to the common ligand, competitive binding versus a detectable natural common ligand and NMR experiments similar to those described above can be performed to confirm that the expansion linker does not interfere with binding of the common ligand to the conserved site and to confirm that the expansion linker is attached to the common ligand oriented towards the specificity site. To determine an expansion linker that provides an optimized orientation for attaching a specificity ligand, more than one expansion linker can be attached to the common ligand and screened.

Once a common ligand-expansion linker has been identified that binds to the conserved site in the correct orientation for attaching a specificity ligand to the expansion linker, a population of bi-ligands is generated. The bi-ligands are generated by attaching potential specificity ligands having reactive groups to the expansion linker at the position on the expansion linker that orients the specificity ligand to the specificity site.

Several advantages are provided by the methods of the invention for identifying a bi-ligand. By starting with a common ligand, the platform for generating a population of bi-ligands is biased toward allowing identification of high affinity ligands because at least part of the binding function is provided by the common ligand, allowing a screen for additional ligands that will bind synergistically and specifically. The attachment of an expansion linker to orient a specificity ligand to a specificity site further enhances the probability of identifying a high affinity, high specificity ligand. The combination of a common ligand with an expansion linker is therefore a useful module for generating a population or library of bi-ligands.

If the bi-ligand is to be further used to generate a bi-target ligand, the second reactive position for attaching a specificity ligand can be attached to a non-ligand, which is a non-binding chemical group, to occupy the position to be subsequently modified with a second specificity ligand. This modification assures that any such bi-ligands generated will likely tolerate the addition of a second ligand at the second position on the expansion linker. Any chemical group can be added to the second specificity ligand attachment point as a non-ligand so long as the group does not bind to the receptor. Examples of such non-ligands include, for example, sugars or polyethylene glycol (of length n=2−10). If the population of specificity ligands is generated from a chemical having similar structure, another representative of the same chemical group can be added to the second specificity site so long as the chemical group does not bind to the receptor and alter its affinity relative to the binding of the common ligand alone. Generally, the non-ligand will not alter the affinity of the common ligand by more than a factor of 10. The non-ligand can be tested to determine that it does not alter binding of a common ligand using the methods described above for screening ligand binding activity.

The population of bi-ligands is screened for binding to a target receptor. Methods of screening for binding of a bi-ligand to a target receptor are well known to those skilled in the art. Methods similar to those described above for identifying a common ligand can be used to determine if a bi-ligand binds to a receptor. For example, a labeled common ligand can be used to screen for a bi-ligand in the population that competitively binds to a receptor. The common ligand can be labeled, for example, with a radioactive or fluorescent label that is readily detectable.

The population of bi-ligands is screened to identify at least one bi-ligand that specifically binds to a receptor. If an initial screen does not allow identification of bi-ligand that binds to a receptor, a larger population of bi-ligands can be generated as described above and screened for binding to the receptor.

Desirable bi-ligands have specificity for a receptor in a receptor family. To determine that a bi-ligand having binding activity to a receptor also has specificity for that receptor, the bi-ligand is screened against at least one other member of the receptor family. The purpose of screening against at least two members of a receptor family is therefore to identify a bi-ligand that has specificity for a receptor in a receptor family.

A ligand exhibiting specificity for a receptor in a receptor family differentially binds to a particular receptor, which is measurably higher than the binding of the ligand to at least one other receptor in the same family. For example, a ligand having 2-fold higher affinity or greater for one receptor over another receptor in the same family is considered to have specificity for binding to that receptor. A ligand having specificity will have 2-fold higher affinity or greater, generally 3- to 5-fold higher affinity or greater, and particularly 5- to 10-fold higher affinity or greater for one receptor compared to at least one other member of the same receptor family. In some cases, the specificity will be 100-fold to 1000-fold higher affinity or greater. Also, a ligand can have specificity for one receptor over two other members, three other members, five other members, ten other members, twenty other members or even all other members of a receptor family. However, it is not necessary to show specificity for one receptor over all other members of the receptor family but, rather, it is sufficient to show that a ligand has specificity for a receptor relative to at least one other member of the receptor family.

Once a bi-ligand has been identified having specificity for a receptor in a receptor family, the bi-ligand can be validated as a likely effective therapeutic agent. For example, if the target receptor is in a pathogenic organism, the bi-ligand can be tested for inhibitory activity in the target organism. Similarly, if the target receptor is in a cell such as a cancer cell, the bi-ligand can be tested for inhibitory activity on an analogous cancer cell line. Particularly desirable bi-ligands are those that kill or slow the growth of a target organism or cell.

Furthermore, a bi-ligand can be further optimized for orienting a common ligand and specificity ligand to their respective sites. For example, a specific bi-ligand can be modified by testing additional expansion linkers in combination with the common ligand and specificity ligand. The common ligand-specificity ligand bridged by various expansion linkers can be screened to identify a bi-ligand comprising a common ligand and specificity ligand tethered by an expansion linker that is optimized for orienting the common ligand and specificity ligand to the conserved site and specificity sites, respectively, of the receptor.

One advantage of using a common ligand as a starting compound for building a population of bi-ligands is that the same population can be used to screen for ligands that bind to other members of the receptor family. Thus, a population of bi-ligands comprising a common ligand are useful for screening for bi-ligands to a variety of receptors in a receptor family. Therefore, the same population of bi-ligands can be screened for binding to a second receptor in a receptor family.

The invention also provides a method for identifying a bi-target ligand to a receptor. The method consists of (a) identifying a first bi-ligand to a first receptor in a receptor family, wherein the bi-ligand comprises a common ligand to a conserved site in a receptor family and a first specificity ligand to the first receptor; (b) identifying a second bi-ligand to a second receptor in a receptor family, wherein the bi-ligand comprises the common ligand and a second specificity ligand to the second receptor; and (c) generating a bi-target ligand comprising the common ligand, the first specificity ligand and the second specificity ligand, whereby the bi-target ligand can bind to the first receptor and the second receptor.

As discussed above, the advantage of using a common ligand as a starting compound to identify bi-ligands having high affinity and specificity is that the population of bi-ligands can be screened against multiple members of a receptor family to identify multiple bi-ligands specific for several to many members of a receptor family. Once at least two bi-ligands exhibiting specificity are identified, the two bi-ligands can be combined into a bi-target ligand having specificity for two different members of a receptor family. When generating a bi-target ligand, the bi-ligands are joined by an expansion linker that provides approximate C2 symmetry. The common ligand and two specificity ligands are linked by an expansion linker that provides C2 symmetry for the two specificity ligands relative to each other. This allows the common ligand, in combination with one of the two specificity ligands, to bind to the respective specific receptor for the respective specificity ligand.

One advantage of generating a bi-target ligand having specificity for two receptors in a receptor family is that the same ligand can be used to inhibit two drug targets in the same organism. A bi-target ligand is more effective at inhibiting a targeted organism because the bi-target ligand inhibits two targets. The use of combination therapy using multi-drug cocktails for treating a variety of diseases has recently grown in clinical applications. Combination therapy is often more effective than treating with a single drug alone. By combining specificity for two ligands into one, a bi-target ligand is generated that essentially functions as two drugs administered as a single drug.

One disadvantage of administering multi-drug cocktails is that clinical trials are initially conducted on individual drugs. Multiple drugs are later combined in additional clinical trials. Often, unpredictable drug interactions or side effects can occur when previously characterized drugs are administered together. An additional advantage of a bi-target ligand is that the effect of both drugs is simultaneously determined, eliminating the possibility of unpredictable drug interactions when the two drugs are combined.

A further advantage of using a bi-target ligand is that a bi-target ligand is inherently refractile to developing drug resistance. One mechanism of drug resistance involves mutations in the target receptor to which a drug binds. By combining two specificity ligands for two target receptors into one ligand, the organism has to develop mutations in both target receptors to overcome the inhibitory activity of a bi-target ligand. Mutation of two different receptor targets to overcome the inhibitory effect of a drug is statistically much less likely to occur than mutation in a single receptor. Therefore, bi-target ligands are inherently retractile to the development of drug resistance during clinical use.

The invention also provides a library of bi-ligands comprising a common ligand to a conserved site in a receptor family and an expansion linker attached to said common ligand, wherein the expansion linker has sufficient length and orientation to direct a second ligand to a specificity site of a receptor in the receptor family to form a module; and a specificity ligand attached to the expansion linker.

The invention additionally provides a population of two or more bi-ligands, comprising: (a) at least one bi-ligand to a first receptor comprising a common ligand to a conserved site in a receptor family and a specificity ligand to a specificity site of the first receptor in the receptor family; and (b) at least one bi-ligand to a second receptor comprising the common ligand and a specificity ligand to a specificity site of said second receptor in the receptor family, wherein the common ligand and the specificity ligand are linked by an expansion linker of sufficient length and orientation to direct the specificity ligand to a specificity site of the receptor.

The invention further provides a bi-target ligand, comprising: (a) a common ligand to a conserved site in a receptor family; (b) a first specificity ligand to a specificity site of a first receptor in the receptor family; and (c) a second specificity ligand to a specificity site of a second receptor in the receptor family, wherein the common ligand and the specificity ligands are linked by an expansion linker of sufficient length and in an orientation directing the first specificity ligand to the specificity site of the first receptor and the second specificity ligand to the specificity site of the second receptor.

A bi-ligand or bi-target ligand can be administered to an individual as a pharmaceutical composition comprising a bi-ligand or bi-target ligand and a pharmaceutically acceptable carrier. Pharmaceutically acceptable carriers are well known in the art and include aqueous solutions such as physiologically buffered saline or other solvents or vehicles such as glycols, glycerol, oils such as olive oil or injectable organic esters.

A pharmaceutically acceptable carrier can contain physiologically acceptable compounds that act, for example, to stabilize the bi-ligand or bi-target ligand or increase the absorption of the agent. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the route of administration of the bi-ligand or bi-target ligand and on the particular physico-chemical characteristics of the specific bi-ligand or bi-target ligand.

One skilled in the art would know that a pharmaceutical composition comprising a bi-ligand or bi-target ligand can be administered to a subject by various routes including, for example, orally or parenterally, such as intravenously (i.v.), intramuscularly, subcutaneously, intraorbitally, intracapsularly, intraperitoneally (i.p.), intracisternally, intra-articularly or by passive or facilitated absorption through the skin using, for example, a skin patch or transdermal iontophoresis, respectively. Thus, a bi-ligand or bi-target ligand can be administered by injection, intubation, orally or topically, the latter of which can be passive, for example, by direct application of an ointment or powder, or active, for example, using a nasal spray or inhalant.

A bi-ligand or bi-target ligand also can be administered as a topical spray, in which case one component of the composition is an appropriate propellant. The pharmaceutical composition also can be incorporated, if desired, into liposomes, microspheres or other polymer matrices (Gregoriadis, Liposome Technology, Vols. I to III, 2nd ed. (CRC Press, Boca Raton Fla. (1993), which is incorporated herein by reference). Liposomes, for example, which consist of phospholipids or other lipids, are nontoxic, physiologically acceptable and metabolizable carriers that are relatively simple to make and administer.

A pharmaceutical composition comprising a bi-ligand or bi-target ligand is administered in an effective dose, which depends on many factors including the age and general health of the subject as well as the route of administration and the number of treatments to be administered. In view of these factors, the skilled artisan would adjust the particular dose so as to obtain an effective dose. The total treatment dose can be administered to a subject as a single dose, either as a bolus or by infusion over a relatively short period of time, or can be administered using a fractionated treatment protocol, in which the multiple doses are administered over a more prolonged period of time.

Although the invention has been described with reference to the examples provided above, it should be understood that various modifications can be made without departing from the spirit of the invention. Accordingly, the invention is limited only by the claims.

I claim:

1. A method for generating a library of bi-ligands, comprising
(a) determining a common ligand to a conserved site in a receptor family;
(b) attaching an expansion linker to said common ligand, wherein said expansion linker has sufficient length and orientation to direct a second ligand to a specificity site of a receptor in said receptor family, to form a module; and
(c) generating a population of bi-ligands comprising a plurality of identical modules attached to variable second ligands.

2. The method of claim 1, further comprising:
(d) screening said population of bi-ligands for binding to a receptor in said receptor family; and
(e) identifying a bi-ligand that binds to and has specificity for said receptor.

3. The method of claim 1, wherein said population comprises three or more bi-ligands.

4. The method of claim 3, wherein said population comprises five or more bi-ligands.

5. The method of claim 1, wherein said receptor is an enzyme selected from the group consisting of kinases, dehydrogenases, oxidoreductases, GTPases, carboxyl transferases, acyl transferases, decarboxylases, transaminases, racemases, methyl transferases, formyl transferases, and α-ketodecarboxylases.

6. The method of claim 1, wherein said receptor family binds a cofactor selected from the group consisting of nicotinamide adenine dinucleotide, nicotinamide adenine dinucleotide phosphate, thiamine pyrophosphate, flavin adenine dinucleotide, flavin mononucleotide, pyridoxal phosphate, coenzyme A, tetrahydrofolate adenosine triphosphate, guanosine triphosphate and S-adenosyl methionine.

7. The method of claim 1, wherein said expansion linker has approximate C2 symmetry.

8. The method of claim 7, wherein said expansion linker has perfect C2 symmetry.

9. A method for identifying a population of bi-ligands to receptors in a receptor family, comprising
(a) determining a common ligand to a conserved site in the receptor family;
(b) attaching an expansion linker to said common ligand, wherein said expansion linker has sufficient length and orientation to direct a second ligand to a specificity site of a receptor in said receptor family, to form a module; and
(c) generating a population of bi-ligands, wherein said bi-ligand comprises said module and a second ligand linked by said expansion linker.

10. The method of claim 9, further comprising:
(d) screening said population of bi-ligands for binding to a receptor in said receptor family;
(e) identifying a bi-ligand that binds to and has specificity for said receptor; and
(f) repeating steps (d) and (e) to identify a bi-ligand that binds to and has specificity for a second receptor in said receptor family.

11. The method of claim 9, wherein said receptor is an enzyme selected from the group consisting of kinases, dehydrogenases, oxidoreductases, GTPases, carboxyl transferases, acyl transferases, decarboxylases, transaminases, racemases, methyl transferases, formyl transferases, and α-ketodecarboxylases.

12. The method of claim 9, wherein said receptor family binds a cofactor selected from the group consisting of nicotinamide adenine dinucleotide, nicotinamide adenine dinucleotide phosphate, thiamine pyrophosphate, flavin adenine dinucleotide, flavin mononucleotide, pyridoxal phosphate, coenzyme A, tetrahydrofolate adenosine triphosphate, guanosine triphosphate and S-adenosyl methionine.

13. The method of claim 9, wherein said expansion linker has approximate C2 symmetry.

14. The method of claim 13, wherein said expansion linker has perfect C2 symmetry.

15. A method for identifying a bi-target ligand to a receptor, comprising
(a) identifying a first bi-ligand to a first receptor in a receptor family, wherein said bi-ligand comprises a common ligand to a conserved site in a receptor family and a first specificity ligand to said first receptor;
(b) identifying a second bi-ligand to a second receptor in said receptor family, wherein said bi-ligand comprises said common ligand and a second specificity ligand to said second receptor; and
(c) generating a bi-target ligand comprising said common ligand, said first specificity ligand and said second specificity ligand, whereby said bi-target ligand can bind to said first receptor and said second receptor.

16. The method of claim 15, wherein said receptor is an enzyme selected from the group consisting of kinases, dehydrogenases, oxidoreductases, GTPases, carboxyl transferases, acyl transferases, decarboxylases, transaminases, racemases, methyl transferases, formyl transferases, and α-ketodecarboxylases.

17. The method of claim 15, wherein said receptor family binds a cofactor selected from the group consisting of nicotinamide adenine dinucleotide, nicotinamide adenine dinucleotide phosphate, thiamine pyrophosphate, flavin adenine dinucleotide, flavin mononucleotide, pyridoxal phosphate, coenzyme A, tetrahydrofolate adenosine triphosphate, guanosine triphosphate and S-adenosyl methionine.

18. The method of claim 15, wherein said expansion linker has approximate C2 symmetry.

19. The method of claim 18, wherein said expansion linker has perfect C2 symmetry.

20. A library of bi-ligands comprising a common ligand to a conserved site in a receptor family and an expansion linker attached to said common ligand, wherein said expansion linker has sufficient length and orientation to direct a second ligand to a specificity site of a receptor in said receptor family to form a module; and a specificity ligand attached to said expansion linker.

21. The library of claim 20, wherein said population comprises three or more bi-ligands.

22. The library of claim 20, wherein said population comprises five or more bi-ligands.

23. The library of claim 20, wherein said receptor is an enzyme selected from the group consisting of kinases, dehydrogenases, oxidoreductases, GTPases, carboxyl transferases, acyl transferases, decarboxylases, transaminases, racemases, methyl transferases, formyl transferases, and α-ketodecarboxylases.

24. The library of claim 20, wherein said receptor family binds a cofactor selected from the group consisting of nicotinamide adenine dinucleotide, nicotinamide adenine dinucleotide phosphate, thiamine pyrophosphate, flavin adenine dinucleotide, flavin mononucleotide, pyridoxal phosphate, coenzyme A, tetrahydrofolate adenosine triphosphate, guanosine triphosphate and S-adenosyl methionine.

25. The library of claim 23, wherein said expansion linker has approximate C2 symmetry.

26. The library of claim 25, wherein said expansion linker has perfect C2 symmetry.

27. A population of two or more bi-ligands, comprising:
(a) at least one bi-ligand to a first receptor comprising a common ligand to a conserved site in a receptor family and a specificity ligand to a specificity site of said first receptor in said receptor family; and
(b) at least one bi-ligand to a second receptor comprising said common ligand and a specificity ligand to a specificity site of said second receptor in said receptor family,
wherein said common ligand and said specificity ligand are linked by an expansion linker of sufficient length and orientation to direct said specificity ligand to a specificity site of said receptor.

28. The population of two or more bi-ligands of claim 27, wherein said receptor is an enzyme selected from the group consisting of kinases, dehydrogenases, oxidoreductases, GTPases, carboxyl transferases, acyl transferases, decarboxylases, transaminases, racemases, methyl transferases, formyl transferases, and α-ketodecarboxylases.

29. The population of two or more bi-ligands of claim 27, wherein said receptor family binds a cofactor selected from the group consisting of nicotinamide adenine dinucleotide, nicotinamide adenine dinucleotide phosphate, thiamine pyrophosphate, flavin adenine dinucleotide, flavin mononucleotide, pyridoxal phosphate, coenzyme A, tetrahydrofolate adenosine triphosphate, guanosine triphosphate and S-adenosyl methionine.

30. The population of two or more bi-ligands of claim 27, wherein said expansion linker has approximate C2 symmetry.

31. The population of two or more bi-ligands of claim 30, wherein said expansion linker has perfect C2 symmetry.

32. A bi-target ligand, comprising:
(a) a common ligand to a conserved site in a receptor family;
(b) a first specificity ligand to a specificity site of a first receptor in said receptor family; and
(c) a second specificity ligand to a specificity site of a second receptor in said receptor family,
wherein said common ligand and said specificity ligands are linked by an expansion linker of sufficient length and in an orientation directing said first specificity ligand to said specificity site of said first receptor and said second specificity ligand to said specificity site of said second receptor.

33. The bi-target ligand of claim 32, wherein said receptor is an enzyme selected from the group consisting of kinases, dehydrogenases, oxidoreductases, GTPases, carboxyl transferases, acyl transferases, decarboxylases, transaminases, racemases, methyl transferases, formyl transferases, and α-ketodecarboxylases.

34. The bi-target ligand of claim 32, wherein said receptor family binds a cofactor selected from the group consisting of nicotinamide adenine dinucleotide, nicotinamide adenine dinucleotide phosphate, thiamine pyrophosphate, flavin adenine dinucleotide, flavin mononucleotide, pyridoxal phosphate, coenzyme A, tetrahydrofolate adenosine triphosphate, guanosine triphosphate and S-adenosyl methionine.

35. The bi-target ligand of claim 32, wherein said expansion linker has approximate C2 symmetry.

36. The bi-target ligand of claim 35, wherein said expansion liner has perfect C2 symmetry.
